(19) United States
(12) Patent Application Publication
(10) Pub. No.: US 2025/0325464 A1
(43) Pub. Date: Oct. 23, 2025

(54) FILM-FORMING COLOR COSMETIC COMPOSITIONS

(71) Applicant: Revlon Consumer Products LLC, New York, NY (US)

(72) Inventors: Sweety Shah, Matawan, NJ (US); Susie Alves Bonavitacola, Clark, NJ (US); Kalliope S. Lordi, Clark, NJ (US)

(21) Appl. No.: 18/868,595

(22) PCT Filed: Jun. 22, 2023
(86) PCT No.: PCT/US2023/068873

§ 371 Date: Nov. 22, 2024

Related U.S. Application Data
(60) Provisional application No. 63/368,385, filed on Jul. 14, 2022.

(51) Int. Cl. A61K 8/58; A61Q 1/06

(52) U.S. Cl. CPC A61K 8/585; A61Q 1/06

(57) ABSTRACT

Cosmetic compositions are described, said compositions comprising, in a cosmetically acceptable medium: (a) at least one polyene; (b) at least one polyether-modified silicone; (c) at least one siloxysilicate resin; and (d) at least one non-emulsifying silicone elastomer. These cosmetic compositions exhibit long wear resistance and improve comfort.

### CROSS REFERENCE TO RELATED APPLICATIONS

This application claims priority to U.S. Provisional Patent Application No. 63/368,385, filed Jul. 14, 2022, entitled “FILM-FORMING COLOR COSMETIC COMPOSITIONS,” which is incorporated by reference herein, in the entirety and for all purposes.

### TECHNICAL FIELD

Cosmetic compositions are described, said compositions comprising, in a cosmetically acceptable medium: (a) at least one polyene; (b) at least one polyether-modified silicone; (c) at least one siloxysilicate resin; and (d) at least one non-emulsifying silicone elastomer. These cosmetic compositions exhibit long wear resistance and improve comfort and are particularly useful as cosmetic compositions for the face, eye, lips, nails or hair and, in particular, as color cosmetic compositions such as lipstick, lip gloss, blush, eyeshadow, mascara, concealer, and the like.

### BACKGROUND OF THE INVENTION

The information provided below is not admitted to be prior art to the present disclosure, but is provided solely to assist the understanding of the reader.

Cosmetic manufacturers seek to provide color cosmetic products that stay on the keratinous surface to which they are applied for extended periods of time. Many consumers desire color cosmetics that will stay on the skin or lips, for example, for at least eight hours, so that when they apply make up in the morning it will stay on their face throughout the workday. Further, the cosmetic should be comfortable to wear with a soft silky feel.

However, color cosmetics applied to keratinous surfaces that are very mobile tend to wear less well. For example, lips are constantly moving as we speak, eat, and drink. Accordingly, lipsticks tend to wear off the lips more quickly than other types of cosmetics. While a consumer might apply foundation make up to the face in the morning expecting that it will remain on the face for the entire day, there are few, if any, lip colors that could be applied to the lips and be expected to remain on the lips for the entire day. Accordingly, cosmetics manufacturers are interested in formulating these types of products because they fill a consumer need gap.

There is a need for color cosmetic products, particularly lip products such as lipsticks, lip glosses, and the like, that provide improved wear such that a consumer can apply the cosmetic to the lips and expect that it will remain for a longer period of time than the standard formulas.

It has been found that the cosmetic compositions of the present disclosure exhibit long wear resistance and improve comfort and, therefore, are particular useful in color cosmetic compositions such as lipstick, lip gloss, blush, eyeshadow, mascara, concealer, and the like.

### SUMMARY

Described herein is a cosmetic composition comprising, in a cosmetically acceptable medium:
(a) at least one polyene;
(b) at least one polyether-modified silicone;
(c) at least one siloxysilicate resin; and
(d) at least one non-emulsifying silicone elastomer.

In an embodiment, the at least one polyene (a) is selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof.

In a particular embodiment, the at least one polyene (a) is selected from the group consisting of polybutene, polyisobutene, polyisoprene, polypentene, polypropylene, and mixtures thereof.

In an embodiment, the at least one polyether-modified silicone (b) is selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof.

In a particular embodiment, the at least one polyether-modified silicone (b) is selected from the group consisting of PEG-3 Dimethicone, PEG-8 Dimethicone, PEG-10 Dimethicone, PEG-12 Dimethicone, and mixtures thereof.

In an embodiment, the at least one siloxysilicate resin (c) is selected from the group consisting of Trimethylsiloxysilicate, Diisostearoyl Trimethylolpropane Siloxysilicate, and mixtures thereof.

In an embodiment, the at least one non-emulsifying silicone elastomer (d) is selected from Vinyl Dimethicone/Methicone Silsesquioxane Crosspolymer.

In an embodiment, the at least one polyene (a) is present at a concentration ranging from about 0.01 to about 5 wt. % based on the total weight of the composition.

In an embodiment, the at least one polyether-modified silicone (b) is present at a concentration ranging from about 0.01 to about 5 wt. % based on the total weight of the composition.

In an embodiment, the at least one siloxysilicate resin (c) is present at a concentration ranging from about 0.1 to about 25 wt. % based on the total weight of the composition.

In an embodiment, the at least one non-emulsifying silicone elastomer (d) is present at a concentration ranging from about 0.1 to about 10 wt. % based on the total weight of the composition.

An embodiment disclosed herein includes a cosmetic composition comprising, in a cosmetically acceptable medium:
(a) at least one polyene selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) at least one siloxysilicate resin selected from the group consisting of Trimethylsiloxysilicate, Diisostearoyl Trimethylolpropane Siloxysilicate, and mixtures thereof; and
(d) at least one non-emulsifying silicone elastomer selected from Vinyl Dimethicone/Methicone Silsesquioxane Crosspolymer.

An embodiment disclosed herein includes a cosmetic composition comprising, in a cosmetically acceptable medium:
(a) at least one polyene selected from the group consisting of polybutene, polyisobutene, polyisoprene, polypentene, polypropylene, and mixtures thereof;
(b) at least one polyether-modified silicone selected from the group consisting of PEG-3 Dimethicone, PEG-8 Dimethicone, PEG-10 Dimethicone, PEG-12 Dimethicone, and mixtures thereof;
(c) at least one siloxysilicate resin selected from the group consisting of Trimethylsiloxysilicate, Diisostearoyl Trimethylolpropane Siloxysilicate, and mixtures thereof; and
(d) at least one non-emulsifying silicone elastomer selected from Vinyl Dimethicone/Methicone Silsesquioxane Crosspolymer.

An embodiment disclosed herein includes a cosmetic composition comprising, in a cosmetically acceptable medium, based on the total weight of the composition:
(a) about 0.01 to 5 wt. % of at least one polyene;
(b) about 0.01 to 5 wt. % of at least one polyether-modified silicone;
(c) about 0.1 to 25 wt. % of at least one siloxysilicate resin; and
(d) about 0.1 to 10 wt. % of at least one non-emulsifying silicone elastomer.

An embodiment disclosed herein includes a cosmetic composition comprising, in a cosmetically acceptable medium, based on the total weight of the composition:
(a) about 0.01 to 5 wt. % of at least one polyene selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) about 0.01 to 5 wt. % of at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) about 0.1 to 25 wt. % of at least one siloxysilicate resin selected from the group consisting of Trimethylsiloxysilicate, Diisostearoyl Trimethylolpropane Siloxysilicate, and mixtures thereof; and
(d) about 0.1 to 10 wt. % of at least one non-emulsifying silicone elastomer selected from Vinyl Dimethicone/Methicone Silsesquioxane Crosspolymer.

An embodiment disclosed herein includes a cosmetic composition comprising, in a cosmetically acceptable medium, based on the total weight of the composition:
(a) about 0.05 to 3 wt. % of at least one polyene;
(b) about 0.05 to 3 wt. % of at least one polyether-modified silicone;
(c) about 1 to 20 wt. % of at least one siloxysilicate resin; and
(d) about 0.5 to 5 wt. % of at least one non-emulsifying silicone elastomer.

An embodiment disclosed herein includes a cosmetic composition comprising, in a cosmetically acceptable medium:
(a) about 0.05 to 3 wt. % of at least one polyene selected from the group consisting of polybutene, polyisobutene, polyisoprene, polypentene, polypropylene, and mixtures thereof;
(b) about 0.05 to 3 wt. % of at least one polyether-modified silicone selected from the group consisting of PEG-3 Dimethicone, PEG-8 Dimethicone, PEG-10 Dimethicone, PEG-12 Dimethicone, and mixtures thereof;
(c) about 1 to 20 wt. % of at least one siloxysilicate resin selected from the group consisting of Trimethylsiloxysilicate, Diisostearoyl Trimethylolpropane Siloxysilicate, and mixtures thereof; and
(d) about 0.5 to 5 wt. % of at least one non-emulsifying silicone elastomer selected from Vinyl Dimethicone/Methicone Silsesquioxane Crosspolymer.

An embodiment includes a method for providing a long-wearing durable fill on the lips comprising applying to the lips a composition as defined herein.

An embodiment includes a method for applying color cosmetics to lips comprising applying to the lips a composition as defined herein.

### DETAILED DESCRIPTION

Embodiments of the present disclosure are discussed in detail below. In describing embodiments, specific terminology is employed for the sake of clarity. However, the disclosure is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the disclosure. While a number of embodiments and features are described herein, it is to be understood that the various features of the disclosure and aspects of embodiments, even if described separately, may be combined unless mutually exclusive or contrary to the specific description. All references cited herein are incorporated by reference as if each had been individually incorporated.

The terms “about” or “approximately” as used herein shall generally mean within 10 percent of a given value. Other than in the operating examples, or where otherwise indicated, all numbers expressing quantities of ingredients and/or reaction conditions are to be understood as being modified in all instances by the term “about”.

The term “at least one” as used herein means one or more of the item to which the term makes reference.

The term “comprising” refers to optional compatible components/steps that can be used provided that the important ingredients/steps are present. The term “comprising” thus encompasses and includes the more restrictive terms “consisting of” and “consisting essentially of”.

“Film former” or “film forming agent” or “film forming polymer” or “film forming resin” as used herein mean a polymer or resin that leaves a film on the substrate to which it is applied, for example, after a solvent accompanying the film former has evaporated, absorbed into and/or dissipated on the substrate.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions. For lip compositions, “long wear” typically means the composition remains on the lips at least about 4 hours up to about 24 hours, and retains rich color even after eating.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion and the one far along described.

“Durable fill” as used herein refers to the substance being able to withstand long wear without damage or changes to the appearance.

### Cosmetically Acceptable Medium

The term “cosmetically acceptable medium” means a carrier for cosmetic use, which carrier delivers the active components to the intended target and which will not cause harm to humans.

The cosmetically acceptable medium is typically a medium comprising water and/or cosmetically acceptable organic solvents including, more particularly, alcohols such as ethyl alcohol, isopropyl alcohol, benzyl alcohol and phenylethyl alcohol; and glycols or glycol ethers such as, for example, glycerin (glycerol), monomethyl, monoethyl and monobutyl ethers of ethylene glycol, propylene glycol (including all possible isomers such as propane-1,2-diol, propane-1,3-diol, propane-2,2-diol or propane-1,1-diol) or its ethers such as, for example, monomethyl ether of propylene glycol, butylene glycol, or dipropylene glycol, as well as alkyl ethers of diethylene glycol such as, for example, monoethyl ether or monobutyl ether of diethylene glycol.

### The Polyene

Polyenes are poly-unsaturated organic compounds that contain at least three alternating double and single carbon-carbon bonds.

The polyene (a) may be selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof.

According to the Personal Care Products Council (“PCPC”) On-Line INFOBASE (resource providing access to personal care and cosmetic collections on ingredients, technical reports, and current news), butene/propylene copolymer (CAS RN 29160-13-2) is a copolymer of butene and propylene monomers.

According to the PCPC On-Line INFOBASE, butylene/ethylene copolymer is a copolymer of butylene and ethylene monomers.

According to the PCPC On-Line INFOBASE, butylene/ethylene/propylene copolymer is a copolymer of butylene, ethylene and propylene monomers.

According to the PCPC On-Line INFOBASE, decene/butene copolymer is a polymer of butene

and decene monomers.

According to the PCPC On-Line INFOBASE, ethylene/octene copolymer (CAS RN 26221-73-8) is a copolymer of ethylene and octene monomers.

According to the PCPC On-Line INFOBASE, ethylene/propylene copolymer (CAS RN 9010-79-1) is the copolymer of ethylene and propylene monomers.

According to the PCPC On-Line INFOBASE, hydrogenated poly(C6-12 olefin) is the end-product of the controlled hydrogenation of the polymer formed by the reaction of C6-12 linear alpha olefin monomers.

According to the PCPC On-Line INFOBASE, hydrogenated poly(C6-14 olefin) is the end-product of the controlled hydrogenation of the polymer formed by the reaction of C6-14 linear alpha olefin monomers.

According to the PCPC On-Line INFOBASE, hydrogenated poly(C6-20 olefin) is the end-product of the controlled hydrogenation of the polymer formed by the reaction of C6-20 linear alpha olefin monomers.

According to the PCPC On-Line INFOBASE, hydrogenated polybutene is the end-product of the controlled hydrogenation of polybutene.

According to the PCPC On-Line INFOBASE, hydrogenated polydecene is the end-product of the controlled hydrogenation of polydecene.

According to the PCPC On-Line INFOBASE, hydrogenated polydodecene is the hydrogenated homopolymer of dodecene.

According to the PCPC On-Line INFOBASE, hydrogenated polyisobutene (CAS RN 68937-10-0) is the polymer that conforms generally to the formula:

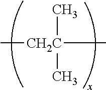

According to the PCPC On-Line INFOBASE, isobutylene/isoprene copolymer (CAS RN 9010-85-9) is a copolymer of isobutylene and isoprene monomers.

According to the PCPC On-Line INFOBASE, isoprene/pentadiene copolymer is a copolymer of isoprene and 1,3-pentadiene monomers.

According to the PCPC On-Line INFOBASE, poly(C4-12 olefin) is a polymer synthesized from C4-12 olefins.

According to the PCPC On-Line INFOBASE, poly(C6-14 olefin) is a polymer synthesized from C6-14 olefins.

According to the PCPC On-Line INFOBASE, poly(C20-28 olefin) is a polymer synthesized from C20-28 olefins.

According to the PCPC On-Line INFOBASE, poly(C30-45 olefin) is a polymer synthesized from C30-45 olefins.

According to the PCPC On-Line INFOBASE, polydecene (CAS RN 25189-70-2) is the polymer formed by the polymerization of decene. It conforms to the formula:

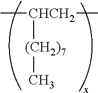

According to the PCPC On-Line INFOBASE, polyethylene (CAS RN 9002-88-4) is a polymer of ethylene monomers that conforms generally to the formula:

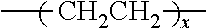

According to the PCPC On-Line INFOBASE, polypropylene (CAS RN 9003-07-0) is a polymer of propylene monomers that conforms generally to the formula:

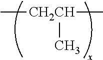

According to the PCPC On-Line INFOBASE, polybutene (CAS RN 9003-28-5) is the polymer formed by the polymerization of a mixture of iso- and normal butenes.

According to the PCPC On-Line INFOBASE, polyisoprene (CAS RN 9003-31-0) is the polymer of isoprene that conforms generally to the formula:

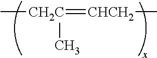

According to the PCPC On-Line INFOBASE, polypentene (CAS RN 9078-70-0) is the polymer formed by the polymerization of pentene that conforms to the formula:

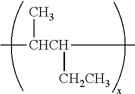

According to the PCPC On-Line INFOBASE, polyisobutene (CAS RN 9003-27-4) is the homopolymer of isobutylene that conforms generally to the formula:

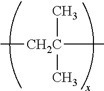

In an embodiment, the polyene (a) is selected from the group consisting of polybutene, polyisobutene, polyisoprene, polypentene, polypropylene, and mixtures thereof.

In one embodiment, the polyene (a) comprises polyisobutene.

Typically, in the cosmetic compositions, the amount of polyene (a) is in the range of about 0.01 to about 5 wt. %, or about 0.05 to about 3 wt. %, or about 0.3 to about 1.5 wt. %, based on the total weight of the composition.

### The Polyether-Modified Silicone

The polyether-modified silicone are silicones containing ethylene oxide (PEG) or propylene oxide (PPG) units.

The polyether-modified silicone (b) may be selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof.

According to the PCPC On-Line INFOBASE, PEG-11 Methyl Ether Dimethicone (CAS RN 68938-54-5) is the methyl ether of a derivative of dimethicone containing an average of 11 moles of ethylene oxide.

According to the PCPC On-Line INFOBASE, PEG-32 Methyl Ether Dimethicone (CAS RN 68938-54-5) is the methyl ether of a derivative of dimethicone containing an average of 32 moles of ethylene oxide.

According to the PCPC On-Line INFOBASE, PEG/PPG-20/22 Butyl Ether Dimethicone (CAS RN 67762-87-2) is the butyl ether of a derivative of dimethicone containing an average of 20 moles of ethylene oxide and 22 moles of propylene oxide.

According to the PCPC On-Line INFOBASE, Cetyl PEG/PPG-10/1 Dimethicone (CAS RN 144243-53-8) is a siloxane polymer prepared by reacting hydrogen dimethicone with hexadecene and the allyl ether of PEG/PPG-10/1.

According to the PCPC On-Line INFOBASE, PEG-9 Polydimethylsiloxyethyl Dimethicone is the siloxane polymer that conforms generally to the formula:

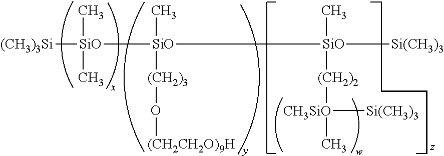

According to the PCPC On-Line INFOBASE, PEG-3 Dimethicone (CAS RN 68937-54-2) is the polyethylene glycol derivative of dimethicone containing an average of 3 moles of ethylene oxide.

According to the PCPC On-Line INFOBASE, PEG-8 Dimethicone (CAS RN 68937-54-2) is the polyethylene glycol derivative of dimethicone containing an average of 8 moles of ethylene oxide.

According to the PCPC On-Line INFOBASE, PEG-10 Dimethicone (CAS RN 68937-54-2) is the polyethylene glycol derivative of dimethicone containing an average of 10 moles of ethylene oxide.

According to the PCPC On-Line INFOBASE, PEG-12 Dimethicone (CAS RN 68937-54-2) is the polyethylene glycol derivative of dimethicone containing an average of 12 moles of ethylene oxide.

In one embodiment, the polyether-modified silicone (b) comprises PEG-3 Dimethicone, PEG-8 Dimethicone, PEG-10 Dimethicone, PEG-12 Dimethicone, and mixtures thereof.

In one embodiment, the polyether-modified silicone (b) comprises PEG-10 Dimethicone.

Typically, in the cosmetic compositions, the amount of polyether-modified silicone (b) is in the range of about 0.01 to about 5 wt. %, or about 0.05 to about 3 wt. %, or about 0.3 to about 1.5 wt. %, based on the total weight of the composition.

### The Siloxysilicate Resin

Siloxysilicate resins, or “Q resins,” are highly cross-linked structures consisting of SiO4/2 units. Siloxysilicate resins of the “MQ” type may also be used.

By way of example, siloxysilicate resins of MQ type, may be comprise:

alkyl siloxysilicates of formula [(R1)₃SiOᵤ₂]ₓ(SiO_{4/2})_{y} (MQ units) in which x and y are integers ranging from 50 to 80, and such that the group R1 represents—a hydrocarbon-based radical containing from 1 to 10 carbon atoms, a phenyl group, a phenylalkyl group or a hydroxyl group; and is preferably an alkyl group containing from 1 to 8 carbon atoms, preferably a methyl group; as silicone resins comprising MQ siloxysilicate units, mention may also be made of phenylalkylsiloxysilicate resins, such as phenylpropyldimethylsiloxysilicate. The preparation of such resins is described in particular in U.S. Pat. No. 5,817,302.

The siloxysilicate resin (c) may be selected from the group consisting of Trimethylsiloxysilicate, Diisostearoyl Trimethylolpropane Siloxysilicate, and mixtures thereof.

According to the PCPC On-Line INFOBASE, Trimethylsiloxysilicate (CAS RN 56275-01-5) is the siloxane polymer that conforms generally to the formula:

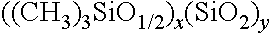

According to the PCPC On-Line INFOBASE, Diisostearoyl Trimethylolpropane Siloxysilicate is the siloxane polymer that conforms generally to the formula:

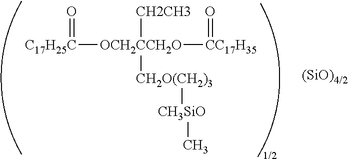

In one embodiment, the siloxysilicate resin (c) comprises Trimethylsiloxysilicate.

Typically, in the cosmetic compositions, the amount of siloxysilicate resin (c) is in the range of about 0.1 to about 25 wt. %, or about 1 to about 20 wt. %, or about 5 to about 18 wt. %, based on the total weight of the composition.

### Non-Emulsifying Silicone Elastomers

The composition of the present disclosure may contain non-emulsifying silicone elastomers.

The term “non-emulsifying” defines silicone elastomers not containing a hydrophilic chain, such as polyoxyalkylene or polyglycerolated units.

The non-emulsifying silicone elastomer (d) may be selected from an elastomeric crosslinked organopolysiloxane that may be obtained by a crosslinking addition reaction of diorganopolysiloxane containing at least one hydrogen linked to silicon and of diorganopolysiloxane containing ethylenically unsaturated groups linked to silicon, especially in the presence of a platinum catalyst; or by dehydrogenation crosslinking coupling reaction between a diorganopolysiloxane containing hydroxyl end groups and a diorganopolysiloxane containing at least one hydrogen linked to silicon, especially in the presence of an organotin compound; or by a crosslinking coupling reaction of a diorganopolysiloxane containing hydroxyl end groups and of a hydrolysable organopolysilane; or by thermal crosslinking of organopolysiloxane, especially in the presence of an organoperoxide catalyst; or by crosslinking of organopolysiloxane via high-energy radiation such as gamma rays, ultraviolet rays or an electron beam.

The elastomeric crosslinked organopolysiloxanes may be conveyed in the form of a gel consisting of an elastomeric organopolysiloxane included in at least one hydrocarbon-based oil and/or one silicone oil. The elastomeric crosslinked organopolysiloxanes may also be in powder form.

Suitable non-emulsifying silicone elastomers for use in the composition of the present invention include, but are not limited to, those sold under the names DC 9040, DC 9041, DC 9509, DC 9505 and DC 9506 by the company Dow Corning, and KSG-6, KSG-8, KSG-10, KSG-14, KSG-15, and KSG-16 by the company Shin-Etsu; SFE-168 and SFE-839 available from GE Silicones; and Gransil SR-SYC available from Grant Industries.

The non-emulsifying silicone elastomer may also be in the form of elastomeric crosslinked organopolysiloxane powder coated with silicone resin, especially with silsesquioxane resin, as described, for example, in U.S. Pat. No. 5,538,793, the entire content of which is herein incorporated by reference. Such elastomers are sold under the names KSP-100, KSP-101, KSP-102, KSP-103, KSP-104 and KSP-105 by the company Shin-Etsu.

Other elastomeric crosslinked organopolysiloxanes in the form of powders include hybrid silicone powders functionalized with fluoroalkyl groups, sold especially under the name KSP-200 by the company Shin-Etsu; hybrid silicone powders functionalized with phenyl groups, sold especially under the name KSP-300 by the company Shin-Etsu.

Additional elastomeric crosslinked organopolysiloxanes in the form of powders include cured silicone powder coated with microfine particles. These particles are described in U.S. Pat. Nos. 5,492,945, 5,756,568 and 5,945,471, the entire contents of which are hereby incorporated by reference. Suitable cured silicone powder coated with microfine particles include, but are not limited to DC9701, available from Dow Corning.

In one embodiment, the non-emulsifying silicone elastomer (d) comprising Vinyl Dimethicone/Methicone Silsesquioxane Crosspolymer.

According to the PCPC On-Line INFOBASE, Vinyl Dimethicone/Methicone Silsesquioxane Crosspolymer is a copolymer of methicone silsesquioxane crosslinked with vinyl dimethylpolysiloxane.

Typically, in the cosmetic compositions, the amount of non-emulsifying silicone elastomer (d) is in the range of about 0.1 to about 10 wt. %, or about 0.5 to about 5 wt. %, or about 0.8 to about 2.5 wt. %, based on the total weight of the composition.

### The Cosmetic Compositions

The cosmetic compositions may include, in a cosmetically acceptable medium:
(a) at least one polyene;
(b) at least one polyether-modified silicone;
(c) at least one siloxysilicate resin; and
(d) at least one non-emulsifying silicone elastomer.
   The at least one polyene (a) may be present at a concentration ranging from about 0.01 to about 5 wt. %, or about 0.05 to about 3 wt. %, or about 0.3 to about 1.5 wt. %, based on the total weight of the composition.

The at least one polyether-modified silicone (b) may be present at a concentration ranging from about 0.01 to about 5 wt. %, or about 0.05 to about 3 wt. %, or about 0.3 to about 1.5 wt. %, based on the total weight of the composition.

The at least one siloxysilicate resin (c) may be present at a concentration ranging from about 0.1 to about 25 wt. %, or about 1 to about 20 wt. %, or about 5 to about 18 wt. %, based on the total weight of the composition.

The at least one non-emulsifying silicone elastomer (d) may be present at a concentration ranging from about 0.1 to about 10 wt. %, or about 0.5 to about 5 wt. %, or about 0.8 to about 2.5 wt. %, based on the total weight of the composition.

In an embodiment, the cosmetic composition comprises, in a cosmetically acceptable medium,
(a) at least one polyene selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) at least one siloxysilicate resin selected from the group consisting of Trimethylsiloxysilicate, Diisostearoyl Trimethylolpropane Siloxysilicate, and mixtures thereof; and
(d) at least one non-emulsifying silicone elastomer selected from Vinyl Dimethicone/Methicone Silsesquioxane Crosspolymer.

In an embodiment, the cosmetic composition comprises, in a cosmetically acceptable medium,
(a) at least one polyene selected from the group consisting of polybutene, polyisobutene, polyisoprene, polypentene, polypropylene, and mixtures thereof;
(b) at least one polyether-modified silicone selected from the group consisting of PEG-3 Dimethicone, PEG-8 Dimethicone, PEG-10 Dimethicone, PEG-12 Dimethicone, and mixtures thereof;
(c) at least one siloxysilicate resin selected from the group consisting of Trimethylsiloxysilicate, Diisostearoyl Trimethylolpropane Siloxysilicate, and mixtures thereof; and
(d) at least one non-emulsifying silicone elastomer selected from Vinyl Dimethicone/Methicone Silsesquioxane Crosspolymer.

In an embodiment, the cosmetic composition comprises, in a cosmetically acceptable medium, based on the total weight of the composition:
(a) about 0.01 to 5 wt. % of at least one polyene;
(b) about 0.01 to 5 wt. % of at least one polyether-modified silicone;
(c) about 0.1 to 25 wt. % of at least one siloxysilicate resin; and
(d) about 0.1 to 10 wt. % of at least one non-emulsifying silicone elastomer.

In an embodiment, the cosmetic composition comprises, in a cosmetically acceptable medium, based on the total weight of the composition:
(a) about 0.01 to 5 wt. % of at least one polyene selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) about 0.01 to 5 wt. % of at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) about 0.1 to 25 wt. % of at least one siloxysilicate resin selected from the group consisting of Trimethylsiloxysilicate, Diisostearoyl Trimethylolpropane Siloxysilicate, and mixtures thereof; and
(d) about 0.1 to 10 wt. % of at least one non-emulsifying silicone elastomer selected from Vinyl Dimethicone/Methicone Silsesquioxane Crosspolymer.

In an embodiment, the cosmetic composition comprises, in a cosmetically acceptable medium, based on the total weight of the composition:
(a) about 0.05 to 3 wt. % of at least one polyene;
(b) about 0.05 to 3 wt. % of at least one polyether-modified silicone;
(c) about 1 to 20 wt. % of at least one siloxysilicate resin; and
(d) about 0.5 to 5 wt. % of at least one non-emulsifying silicone elastomer.

In an embodiment, the cosmetic composition comprises, in a cosmetically acceptable medium, based on the total weight of the composition:
(a) about 0.05 to 3 wt. % of at least one polyene selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) about 0.05 to 3 wt. % of at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) about 1 to 20 wt. % of at least one siloxysilicate resin selected from the group consisting of Trimethylsiloxysilicate, Diisostearoyl Trimethylolpropane Siloxysilicate, and mixtures thereof; and
(d) about 0.5 to 5 wt. % of at least one non-emulsifying silicone elastomer selected from Vinyl Dimethicone/Methicone Silsesquioxane Crosspolymer.

In an embodiment, the cosmetic composition comprises, in a cosmetically acceptable medium,
(a) about 0.05 to 3 wt. % of at least one polyene selected from the group consisting of polybutene, polyisobutene, polyisoprene, polypentene, polypropylene, and mixtures thereof;
(b) about 0.05 to 3 wt. % of at least one polyether-modified silicone selected from the group consisting of PEG-3 Dimethicone, PEG-8 Dimethicone, PEG-10 Dimethicone, PEG-12 Dimethicone, and mixtures thereof;
(c) about 1 to 20 wt. % of at least one siloxysilicate resin selected from the group consisting of Trimethylsiloxysilicate, Diisostearoyl Trimethylolpropane Siloxysilicate, and mixtures thereof; and
(d) about 0.5 to 5 wt. % of at least one non-emulsifying silicone elastomer selected from Vinyl Dimethicone/Methicone Silsesquioxane Crosspolymer.

In an embodiment, the cosmetic composition comprises, in a cosmetically acceptable medium, based on the total weight of the composition:
(a) about 0.3 to 1.5 wt. % of at least one polyene;
(b) about 0.3 to 1.5 wt. % of at least one polyether-modified silicone;
(c) about 5 to 18 wt. % of at least one siloxysilicate resin; and
(d) about 0.8 to 2.5 wt. % of at least one non-emulsifying silicone elastomer.

In an embodiment, the cosmetic composition comprises, in a cosmetically acceptable medium, based on the total weight of the composition:
(a) about 0.3 to 1.5 wt. % of at least one polyene selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) about 0.3 to 1.5 wt. % of at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) about 5 to 18 wt. % of at least one siloxysilicate resin selected from the group consisting of Trimethylsiloxysilicate, Diisostearoyl Trimethylolpropane Siloxysilicate, and mixtures thereof; and
(d) about 0.8 to 2.5 wt. % of at least one non-emulsifying silicone elastomer selected from Vinyl Dimethicone/Methicone Silsesquioxane Crosspolymer.

In an embodiment, the cosmetic composition comprises, in a cosmetically acceptable medium,
(a) about 0.3 to 1.5 wt. % of at least one polyene selected from the group consisting of polybutene, polyisobutene, polyisoprene, polypentene, polypropylene, and mixtures thereof;
(b) about 0.3 to 1.5 wt. % of at least one polyether-modified silicone selected from the group consisting of PEG-3 Dimethicone, PEG-8 Dimethicone, PEG-10 Dimethicone, PEG-12 Dimethicone, and mixtures thereof;
(c) about 5 to 18 wt. % of at least one siloxysilicate resin selected from the group consisting of Trimethylsiloxysilicate, Diisostearoyl Trimethylolpropane Siloxysilicate, and mixtures thereof; and
(d) about 0.8 to 2.5 wt. % of at least one non-emulsifying silicone elastomer selected from Vinyl Dimethicone/Methicone Silsesquioxane Crosspolymer.

### Other Ingredients

The cosmetic composition of the present disclosure may contain additional optional ingredients. Additional optional ingredients may include, for example, pigments, particulate fillers, oils, viscosity modifiers, vitamins and antioxidants, additional botanical extracts, and mixtures thereof. Optional ingredients useful herein are described, for example, in the International Cosmetic Ingredient Dictionary and Handbook by CTFA, 10th Edition (2004).

### Pigments

The cosmetic composition of the present disclosure may comprise about 0.05-40 wt. %, or about 0.1-30 wt. %, or about 0.5-25 wt. %, based on the total weight of the composition of one or more pigments which may be organic or inorganic. Examples of organic pigment families that may be used herein include azo, (including monoazo and diazo), fluoran, xanthene, indigoid, triphenylmethane, anthroquinone, pyrene, pyrazole, quinoline, quinoline, or metallic salts thereof. Preferred are D&C colors, FD&C colors, or Lakes of D&C or FD&C colors. The term “D&C” means drug and cosmetic colors that are approved for use in drugs and cosmetics by the FDA. The term “FD&C” means food, drug, and cosmetic colors which are approved for use in foods, drugs, and cosmetics by the FDA. Certified D&C and FD&C colors are listed in 21 CFR 74.101 et seq. and include the FD&C colors Blue 1, Blue 2, Green 3, Orange B, Citrus Red 2, Red 3, Red 4, Red 40, Yellow 5, Yellow 6, Blue 1, Blue 2; Orange B, Citrus Red 2; and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Suitable Lakes of D&C and FD&C colors are defined in 21 CFR 82.51. Particularly preferred are Lakes formed by the reaction of the organic pigment with a metallic salt such as aluminum, calcium, zirconium, barium, and the like. Suitable reds include pigments from the monoazo, disazo, fluoran, xanthene, or indigoid families or Lakes thereof, such as Red 4, 6, 7, 17, 21, 22, 27, 28, 30, 31, 33, 34, 36, and Red 40. Also suitable are Lakes of such red pigments. Typically, the metal salts are aluminum, barium, and the like

Suitable yellows include those where the yellow pigment is a pyrazole, monoazo, fluoran, xanthene, quinoline, or salt thereof, such as Yellow 5, 6, 7, 8, 10, and 11, as well as Lakes of such yellow pigments.

Suitable violets include those from the anthroquinone family, such as Violet 2 and Lakes thereof. Examples of orange pigments are Orange 4, 5, 10, 11, or Lakes thereof.

Suitable inorganic pigments include iron oxides such as red, blue, black, green, and yellow; titanium dioxide, bismuth oxychloride, and the like. Preferred are iron oxides. The iron oxides may be treated with hydrophobic agents such as silicone, lecithin, mineral oil, or similar materials, will cause the pigment to be hydrophobic or lipophilic in nature, exhibiting an affinity for oily phase ingredients.

### Particulate Fillers

The composition may contain one or more particulate fillers, which are generally non-pigmentitious powdery materials. If so, suggested ranges are about 0.001-40 wt. %, or about 0.05-35 wt. %, or about 0.1-30 wt. % based on the total weight of the composition. Typically, the particulate fillers have particle sizes ranging from about 0.02 to 100, or from about 0.5 to 100, microns. Suitable particulate fillers include titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silk powder, silica, talc, mica, zinc laurate, zinc myristate, zinc rosinate, alumina, attapulgite, calcium carbonate, calcium silicate, dextran, kaolin, nylon, silica silylate, sericite, soy flour, tin oxide, titanium hydroxide, trimagnesium phosphate, walnut shell powder, or mixtures thereof. The above-mentioned powders may be surface treated with lecithin, amino acids, mineral oil, silicone oil or various other agents either alone or in combination, which coat the powder surface and render the particles more lipophilic in nature.

### Oils

The composition may contain one or more oils, and if so in ranges from about 0.1-95 wt. %, or about 5-80 wt. %, or about 10-75 wt. % based on the total weight of the composition. The term “oil” means a material that is a pourable liquid at room temperature. A variety of such oils are suitable including volatile oils, nonvolatile oils, and mixtures thereof.

### Volatile Oils

The term “volatile” means that the oil has a measurable vapor pressure, or a vapor pressure of at least about 2 mm. of mercury at 20° C. The term “non-volatile” means that the oil has a vapor pressure of less than about 2 mm. of mercury at 20° C. Suitable volatile oils generally have a viscosity of about 0.5 to 10 centipoises at 25° C. and include linear silicones, cyclic silicones, paraffinic hydrocarbons, or mixtures thereof.

Linear and cyclic volatile silicones are available from various commercial and comprise octamethylcyclotetrasiloxane, decamethylcyclopentasiloxane, hexamethyldisiloxane, and mixtures thereof.

Also suitable as the volatile oil are various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C8-20 isoparaffins. Particularly suitable volatile paraffinic hydrocarbons have a molecular weight of 70-225, preferably 160 to 190 and a boiling point range of 30 to 320, preferably 60-260° C., and a viscosity of less than 10 cs. at 25° C. Particularly suitable C8-20 isoparaffins include C12 isoparaffin (isododecane).

### Non-Volatile Oils

The composition may also comprise one or more non-volatile liquid oils such as silicones, esters, and the like. In the case where it is desired to make long wearing cosmetic products, if the non-volatile oils are too heavy or greasy it may hamper the long wearing characteristics of the invention. In such a case, the viscosity of the nonvolatile oils, if present, should range from about 11-1000, preferably less than 100 centipoises, most preferably less than about 50 centipoises at 25° C. Examples of such oils include polyalkylsiloxanes, polyarylsiloxanes, and polyethersiloxanes. Nonlimiting examples of such non-volatile silicones include dimethicone, phenyl trimethicone, and so on.

Also suitable are lower viscosity organic liquids including saturated or unsaturated, substituted or unsubstituted branched or linear or cyclic organic compounds that are liquid under ambient conditions.

If desired, the claimed composition may contain one or more nonvolatile oils. Such oils generally have a viscosity of greater than 10 centipoises at 25° C., and may range in viscosity up to 1,000,000 centipoises at 25° C. Such nonvolatile oils are preferably liquid at room temperature (e.g. 25° C.), and include those set forth below. In the event long-wearing or transfer resistant compositions are desired, if non-volatile oils are present, they are preferably of lower viscosity, e.g. ranging from about 10 to 100,000, preferably 10-50,000, more preferably 10-1000 centipoise at room temperature. Further examples of non-volatile oils include those set forth below.

### Esters

Suitable esters are mono-, di-, and triesters. The composition may comprise one or more esters selected from the group, or mixtures listed below.

### Monoesters

Monoesters are defined as esters formed by the reaction of a monocarboxylic acid having the formula R—COOH, wherein R is a straight or branched chain saturated or unsaturated alkyl having 2 to 30 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups, and in one preferred embodiment of the invention the acid is an alpha hydroxy acid. Either one or both of the acid or alcohol may be a “fatty” acid or alcohol, ie. may have from about 6 to 22 carbon atoms. Examples of monoester oils that may be used in the compositions of the invention include hexyldecyl benzoate, hexyl laurate, hexadecyl isostearate, hexydecyl laurate, hexyldecyl octanoate, hexyldecyl oleate, hexyldecyl palmitate, hexyldecyl stearate, hexyldodecyl salicylate, hexyl isostearate, butyl acetate, butyl isostearate, butyl oleate, butyl octyl oleate, cetyl palmitate, ceyl octanoate, cetyl laurate, cetyl lactate, isostearyl isononanoate, cetyl isononanoate, cetyl stearate, stearyl lactate, stearyl octanoate, stearyl heptanoate, stearyl stearate, and so on. It is understood that in the above nomenclature, the first term indicates the alcohol and the second term indicates the acid in the reaction, i.e. stearyl octanoate is the reaction product of stearyl alcohol and octanoic acid.

### Diesters

Suitable diesters that may be used in the compositions of the invention are the reaction product of a dicarboxylic acid and an aliphatic or aromatic alcohol. The dicarboxylic acid may contain from 2 to 30 carbon atoms, and may be in the straight or branched chain, saturated or unsaturated form. The dicarboxylic acid may be substituted with one or more hydroxyl group. The aliphatic or aromatic alcohol may also contain 2 to 30 carbon atoms, and may be in the straight or branched chain, saturated, or unsaturated form. The aliphatic or aromatic alcohol may be substituted with one or more substituents such as hydroxyl. Preferably, one or more of the acid or alcohol is a fatty acid or alcohol, i.e. contains 14-22 carbon atoms. The dicarboxylic acid may also be an alpha hydroxy acid. Examples of diester oils that may be used in the compositions of the invention include diisostearyl malate, neopentyl glycol dioctanoate, dibutyl sebacate, di-C 12-13 alkyl malate, dicetearyl dimer dilinoleate, dicetyl adipate, diisocetyl adipate, diisononyl adipate, diisostearyl dimer dilinoleate, disostearyl fumarate, diisostearyl malate, and so on.

### Triesters

Suitable triesters comprise the reaction product of a tricarboxylic acid and an aliphatic or aromatic alcohol. As with the mono- and diesters mentioned above, the acid and alcohol contain 2 to 30 carbon atoms, and may be saturated or unsatured, straight or branched chain, and may be substituted with one or more hydroxyl groups. Preferably, one or more of the acid or alcohol is a fatty acid or alcohol containing 14 to 22 carbon atoms. Examples of triesters include triarachidin, tributyl citrate, triisostearyl citrate, tri C12-13 alkyl citrate, tricaprylin, tricaprylyl citrate, tridecyl behenate, trioctyldodecyl citrate, tridecyl behenate, tridecyl cocoate, tridecyl isononanoate, and so on.

### Hydrocarbon Oils

It may be desirable to incorporate one or more non-volatile hydrocarbon oils into the claimed composition. The term “nonvolatile” means that the oil has a vapor pressure of less than about 2 mm. of mercury at 20° C.

Suitable nonvolatile hydrocarbon oils include isoparaffins and olefins having greater than 20 carbon atoms. Examples of such hydrocarbon oils include C 24-28 olefins, C30-45 olefins, C20-40 isoparaffins, hydrogenated polyisobutene, mineral oil, pentahydrosqualene, squalene, squalane, and mixtures thereof.

### Glyceryl Esters of Fatty Acids

The nonvolatile oil may also comprise naturally occurring glyceryl esters of fatty acids, or triglycerides. Both vegetable and animal sources may be used. Examples of such oils include castor oil, lanolin oil, C 10-18 triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable as the oil are synthetic or semi-synthetic glyceryl esters, e.g. fatty acid mono-, di-, and triglycerides which are natural fats or oils that have been modified, for example, acetylated castor oil, or mono-, di- or triesters of polyols such as glyceryl stearate, diglyceryl diiosostearate, polyglyceryl-4 isostearate, polyglyceryl-6 ricinoleate, glyceryl dioleate, glyceryl diisotearate, glyceryl trioctanoate, diglyceryl distearate, glyceryl linoleate, glyceryl myristate, glyceryl isostearate, PEG castor oils, PEG glyceryl oleates, PEG glyceryl stearates, PEG glyceryl tallowates, and so on.

### Non-Volatile Silicones

Non-volatile silicone oils, both water soluble and water insoluble, are also suitable for use as the non-volatile oil. Such silicones preferably have a viscosity ranging from about 10 to 600,000 centistokes, preferably 20 to 100,000 centistokes at 25° C. Suitable water insoluble silicones include amine functional silicones such as amodimethicone; phenyl substituted silicones such as bisphenylhexamethicone, phenyl trimethicone, or polyphenylmethylsiloxane; dimethicone, alkyl substituted dimethicones, and mixtures thereof.

Water soluble, non-film forming silicones such as dimethiconol, and the like may be used.

### Viscosity Modifiers

It may also be desirable to include one or more viscosity modifiers or thickeners in the composition. Suggested ranges of such viscosity modifiers are about 0.01-60 wt. %, or about 0.05-50 wt. %, or about 0.1-45 wt. %, based on the total weight of the composition.

One type of viscosity modifier includes natural or synthetic montmorillonite minerals such as hectorite, bentonite, and quaternized derivatives thereof which are obtained by reacting the minerals with a quaternary ammonium compound, such as stearalkonium bentonite, hectorites, quaternized hectorites such as Quaternium-18 hectorite, attapulgite, carbonates such as propylene carbonate, bentones, and the like. Particularly suitable is Disteardimonium Hectorite.

Also suitable as the viscosity modifier are various polymeric compounds known in the art as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain (C8-C30) alkyl groups such as dodecyl, hexadecyl, or octadecyl; alkylaryl groups such as octylphenyl or nonyphenyl.

Another type of viscosity modifier that may be used in the compositions are silicas, silicates, silica silylate, and derivatives thereof. These silicas and silicates are generally found in the particulate form. Particularly suitable is silica.

The viscosity modifiers may also be water soluble or water insoluble (e.g. oil soluble) and form part of the oil phase or the water phase.

Also suitable as viscosity modifiers are one or more waxes. A variety of waxes are suitable including animal, vegetable, mineral, or silicone waxes. Generally such waxes have a melting point ranging from about 28 to 125° C., preferably about 30 to 100° C. Examples of waxes include acacia, beeswax, ceresin, cetyl esters, flower wax, citrus wax, carnauba wax, jojoba wax, Japan wax, polyethylene, microcrystalline, rice bran, lanolin wax, mink, montan, bayberry, ouricury, ozokerite, palm kernel wax, paraffin, avocado wax, apple wax, shellac wax, clary wax, spent grain wax, candelilla, grape wax, and polyalkylene glycol derivatives thereof such as PEG6-20 beeswax, or PEG-12 carnauba wax.

Also suitable are various types of silicone waxes, referred to as alkyl silicones, which are polymers that comprise repeating dimethylsiloxy units in combination with one or more methyl-long chain (C8-C30) alkyl siloxy units wherein the long chain alkyl is generally a fatty chain that provides a wax-like characteristic to the silicone. Such silicones include, but are not limited to stearoxydimethicone, behenoxy dimethicone, stearyl dimethicone, cetearyl dimethicone, and so on.

### Vitamins and Antioxidants

The compositions of the invention may contain vitamins and antioxidants. If so, suggested ranges of vitamins and antioxidants are about 0.001-10 wt. %, or about 0.01-8 wt. %, or about 0.05-5 wt. %, based on the total weight of the composition. Suitable vitamins include ascorbic acid and derivatives thereof, the B vitamins such as thiamine, riboflavin, pyridoxin, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

### Other Botanical Extracts

It may be desirable to include one or more additional botanical extracts in the compositions. If so, suggested ranges are from about 0 to 10 wt. %, or about 0.0005 to 8 wt. %, or about 0.001 to 5 wt. %, based on the total weight of the composition. Suitable botanical extracts include extracts from plants (herbs, roots, flowers, fruits, seeds) such as flowers, fruits, vegetables, and so on, including acacia (dealbata, famesiana, senegal), acer saccharinum (sugar maple), acidopholus, acorus, aesculus, agaricus, agave, agrimonia, algae, aloe, citrus, brassica, cinnamon, orange, apple, blueberry, cranberry, peach, pear, lemon, lime, pea, seaweed, green tea, chamomile, willowbark, mulberry, poppy, and those set forth in the International Cosmetic Ingredient Dictionary and Handbook by CTFA, 10^{th} Edition (2004).

In an embodiment, the cosmetic composition comprises, in a cosmetically acceptable medium,
(a) at least one polyene selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) at least one siloxysilicate resin selected from the group consisting of Trimethylsiloxysilicate, Diisostearoyl Trimethylolpropane Siloxysilicate, and mixtures thereof;
(d) at least one non-emulsifying silicone elastomer selected from Vinyl Dimethicone/Methicone Silsesquioxane Crosspolymer;
(e) at least one pigment; and
(f) optionally one additional ingredient selected from the group consisting of particulate fillers, oils, viscosity modifiers, vitamins and antioxidants, additional botanical extracts, and mixtures thereof.

In an embodiment, the cosmetic composition comprises, in a cosmetically acceptable medium,
(a) at least one polyene selected from the group consisting of polybutene, polyisobutene, polyisoprene, polypentene, polypropylene, and mixtures thereof;
(b) at least one polyether-modified silicone selected from the group consisting of PEG-3 Dimethicone, PEG-8 Dimethicone, PEG-10 Dimethicone, PEG-12 Dimethicone, and mixtures thereof;
(c) at least one siloxysilicate resin selected from the group consisting of Trimethylsiloxysilicate, Diisostearoyl Trimethylolpropane Siloxysilicate, and mixtures thereof;
(d) at least one non-emulsifying silicone elastomer selected from Vinyl Dimethicone/Methicone Silsesquioxane Crosspolymer;
(e) at least one pigment; and
(f) optionally one additional ingredient selected from the group consisting of particulate fillers, oils, viscosity modifiers, vitamins and antioxidants, additional botanical extracts, and mixtures thereof.

In an embodiment, the cosmetic composition comprises, in a cosmetically acceptable medium, based on the total weight of the composition:
(a) about 0.01 to 5 wt. % of at least one polyene;
(b) about 0.01 to 5 wt. % of at least one polyether-modified silicone;
(c) about 0.1 to 25 wt. % of at least one siloxysilicate resin;
(d) about 0.1 to 10 wt. % of at least one non-emulsifying silicone elastomer;
(e) about 0.05 to 40 wt. % of at least one pigment; and
(f) optionally one additional ingredient selected from the group consisting of particulate fillers, oils, viscosity modifiers, vitamins and antioxidants, additional botanical extracts, and mixtures thereof.

In an embodiment, the cosmetic composition comprises, in a cosmetically acceptable medium, based on the total weight of the composition:
(a) about 0.01 to 5 wt. % of at least one polyene selected the group consisting of from butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) about 0.01 to 5 wt. % of at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) about 0.1 to 25 wt. % of at least one siloxysilicate resin selected from the group consisting of Trimethylsiloxysilicate, Diisostearoyl Trimethylolpropane Siloxysilicate, and mixtures thereof;
(d) about 0.1 to 10 wt. % of at least one non-emulsifying silicone elastomer selected from Vinyl Dimethicone/Methicone Silsesquioxane Crosspolymer;
(e) about 0.05 to 40 wt. % of at least one pigment; and
(f) optionally one additional ingredient selected from the group consisting of particulate fillers, oils, viscosity modifiers, vitamins and antioxidants, additional botanical extracts, and mixtures thereof.

In an embodiment, the cosmetic composition comprises, in a cosmetically acceptable medium, based on the total weight of the composition:
(a) about 0.05 to 3 wt. % of at least one polyene;
(b) about 0.05 to 3 wt. % of at least one polyether-modified silicone;
(c) about 1 to 20 wt. % of at least one siloxysilicate resin;
(d) about 0.5 to 5 wt. % of at least one non-emulsifying silicone elastomer;
(e) about 0.01 to 30 wt. % of at least one pigment; and
(f) optionally one additional ingredient selected from the group consisting of particulate fillers, oils, viscosity modifiers, vitamins and antioxidants, additional botanical extracts, and mixtures thereof.

In an embodiment, the cosmetic composition comprises, in a cosmetically acceptable medium, based on the total weight of the composition:
(a) about 0.05 to 3 wt. % of at least one polyene selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) about 0.05 to 3 wt. % of at least one polyether-modified silicone selected the group consisting of from PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) about 1 to 20 wt. % of at least one siloxysilicate resin selected from the group consisting of Trimethylsiloxysilicate, Diisostearoyl Trimethylolpropane Siloxysilicate, and mixtures thereof;
(d) about 0.5 to 5 wt. % of at least one non-emulsifying silicone elastomer selected from Vinyl Dimethicone/Methicone Silsesquioxane Crosspolymer;
(e) about 0.01 to 30 wt. % of at least one pigment; and
(f) optionally one additional ingredient selected from the group consisting of particulate fillers, oils, viscosity modifiers, vitamins and antioxidants, additional botanical extracts, and mixtures thereof.

In an embodiment, the cosmetic composition comprises, in a cosmetically acceptable medium,
(a) about 0.05 to 3 wt. % of at least one polyene selected from the group consisting of polybutene, polyisobutene, polyisoprene, polypentene, polypropylene, and mixtures thereof;
(b) about 0.05 to 3 wt. % of at least one polyether-modified silicone selected from the group consisting of PEG-3 Dimethicone, PEG-8 Dimethicone, PEG-10 Dimethicone, PEG-12 Dimethicone, and mixtures thereof;
(c) about 1 to 20 wt. % of at least one siloxysilicate resin selected from the group consisting of Trimethylsiloxysilicate, Diisostearoyl Trimethylolpropane Siloxysilicate, and mixtures thereof;
(d) about 0.5 to 5 wt. % of at least one non-emulsifying silicone elastomer selected from Vinyl Dimethicone/Methicone Silsesquioxane Crosspolymer;
(e) about 0.01 to 30 wt. % of at least one pigment; and
(f) optionally one additional ingredient selected from the group consisting of particulate fillers, oils, viscosity modifiers, vitamins and antioxidants, additional botanical extracts, and mixtures thereof.

In an embodiment, the cosmetic composition comprises, in a cosmetically acceptable medium, based on the total weight of the composition:
(a) about 0.3 to 1.5 wt. % of at least one polyene;
(b) about 0.3 to 1.5 wt. % of at least one polyether-modified silicone;
(c) about 5 to 18 wt. % of at least one siloxysilicate resin;
(d) about 0.8 to 2.5 wt. % of at least one non-emulsifying silicone elastomer;
(e) about 0.5 to 25 wt. % of at least one pigment; and
(f) optionally one additional ingredient selected from the group consisting of particulate fillers, oils, viscosity modifiers, vitamins and antioxidants, additional botanical extracts, and mixtures thereof.

In an embodiment, the cosmetic composition comprises, in a cosmetically acceptable medium, based on the total weight of the composition:
(a) about 0.3 to 1.5 wt. % of at least one polyene selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) about 0.3 to 1.5 wt. % of at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) about 5 to 18 wt. % of at least one siloxysilicate resin selected from the group consisting of Trimethylsiloxysilicate, Diisostearoyl Trimethylolpropane Siloxysilicate, and mixtures thereof;
(d) about 0.8 to 2.5 wt. % of at least one non-emulsifying silicone elastomer selected from Vinyl Dimethicone/Methicone Silsesquioxane Crosspolymer;
(e) about 0.5 to 25 wt. % of at least one pigment; and
(f) optionally one additional ingredient selected from the group consisting of particulate fillers, oils, viscosity modifiers, vitamins and antioxidants, additional botanical extracts, and mixtures thereof.

In an embodiment, the cosmetic composition comprises, in a cosmetically acceptable medium,
(a) about 0.3 to 1.5 wt. % of at least one polyene selected from the group consisting of polybutene, polyisobutene, polyisoprene, polypentene, polypropylene, and mixtures thereof;
(b) about 0.3 to 1.5 wt. % of at least one polyether-modified silicone selected from the group consisting of PEG-3 Dimethicone, PEG-8 Dimethicone, PEG-10 Dimethicone, PEG-12 Dimethicone, and mixtures thereof;
(c) about 5 to 18 wt. % of at least one siloxysilicate resin selected from the group consisting of Trimethylsiloxysilicate, Diisostearoyl Trimethylolpropane Siloxysilicate, and mixtures thereof;
(d) about 0.8 to 2.5 wt. % of at least one non-emulsifying silicone elastomer selected from Vinyl Dimethicone/Methicone Silsesquioxane Crosspolymer;
(e) about 0.5 to 25 wt. % of at least one pigment; and
(f) optionally one additional ingredient selected from the group consisting of particulate fillers, oils, viscosity modifiers, vitamins and antioxidants, additional botanical extracts, and mixtures thereof.

### Methods of Applying the Cosmetic Compositions

An embodiment includes a method for providing a long-wearing durable fill on the lips comprising applying to the lips a composition as defined herein.

Another embodiment includes a method for applying color cosmetics to lips comprising applying to the lips a composition as defined herein.

### EXAMPLES

The following examples are given to provide a person skilled in the art with a sufficiently clear and complete explanation of the present disclosure, but should not be considered as limiting of the essential aspects of its subject, as set out in the preceding portions of this description. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. “About” is understood to mean that a given value could vary by 10%. Numerical ranges expressed in the format “from x to y” are understood to include x and y. When for a specific feature multiple preferred ranges are described in the format “from x to y”, it is understood that all ranges combining the different endpoints are also contemplated.

The compositions of Table 1 were prepared by placing the ingredients listed in the table in a container having a homogenizer mill attached and heating to about 90-95° C. The compositions were milled until complete homogenization and then cooled to room temperature while milling. Finally, the compositions were kept undisturbed for more than 24 h.

TABLE 1
Cosmetic compositions (wt. % based on
the total weight of the composition)

| Ingredients | C1 | CE1 | CE2 | CE3 | CE4 | CE5 | CE6 | CE7 |
|---|---|---|---|---|---|---|---|---|
| Polyisobutene | 0.5 | 0.5 | 0.5 | 0.5 | 0.5 | — | 0.5 | 0.5 |
| PEG-10 | 0.5 | 0.5 | — | — | 0.5 | 0.5 | — | 0.5 |
| Dimethicone | | | | | | | | |
| Trimethyl- | 9 | 9 | 9 | 9 | — | 9 | 9 | 9 |
| siloxysilicate | | | | | | | | |
| (pre-solubilized | | | | | | | | |
| in 10% of | | | | | | | | |
| Isododecane) | | | | | | | | |
| Vinyl | 1.5 | — | 1.5 | 1.5 | 1.5 | 1.5 | 1.5 | — |
| Dimethicone/ | | | | | | | | |
| Methicone | | | | | | | | |
| Silsesquioxane | | | | | | | | |
| Crosspolymer | | | | | | | | |
| Silica Silylate | — | 1.5 | — | — | — | — | — | — |
| PEG-8 Distearate | — | — | 0.25 | 2.0 | — | — | — | — |
| Pigments, | up to 100 wt. % | | | | | | | |
| particulate | | | | | | | | |
| fillers, oils | | | | | | | | |
| and viscosity | | | | | | | | |
| modifiers | | | | | | | | |

C1 is a composition according to the present disclosure and CE1 to CE7 are Comparative Experiments:
in Comparative Experiment 1 (CE1), Vinyl Dimethicone/Methicone Silsesquioxane Crosspolymer was replaced with Silica Silylate;
in Comparative Experiment 2 (CE2), PEG-10 Dimethicone was replaced with 0.25 wt. % of PEG-8 Distearate;
in Comparative Experiment 3 (CE3), PEG-10 Dimethicone was replaced with 2 wt. % of PEG-8 Distearate;
in Comparative Experiment 4 (CE4), Trimethylsiloxysilicate was not added;
in Comparative Experiment 5 (CE5), Polyisobutene was not added;
in Comparative Experiment 6 (CE6), PEG-10 Dimethicone was not added; and
in Comparative Experiment 7 (CE7), Vinyl Dimethicone/Methicone Silsesquioxane Crosspolymer was not added.

To assess the wear durable fill, a visual inspection of each composition was carried out.

In order to present long wear properties, the compositions should exhibit a clear barrier (film). The result of this visual assessment is displayed in Table 2.

TABLE 2
Visual assessment of the cosmetic compositions

| Composition | Visual inspection |
|---|---|
| C1 | A clear gelled barrier (layer) was formed on top of the |
| | composition. If some product was removed from the layer, |
| | it filled back in, displaying a self-levelling effect |
| CE1 | No barrier was formed |
| CE2 | Swirling/streaks were observed |
| CE3 | Swirling/streaks were observed. In addition, the |
| | composition was tacky and with a heavy feeling |
| CE4 | No barrier was formed |
| CE5 | Swirling/streaks were observed |
| CE6 | No barrier was formed |
| CE7 | Very faint barrier was formed at the top of the mixture, |
| | which did not provide the same wear properties as C1 |

Results show that the gelled barrier was not formed in the Comparative Examples. The self-leveling effect makes the composition according to the present disclosure a suitable option for long wear cosmetic products with a lightweight comfortable feel. Self-leveling as used herein is the ability to remove the substance and for the substance to fill back in and level out so that it looks the same as when it was applied.

Modifications that do not affect, alter, change or modify the essential aspects of the compositions and methods described above, are included within the scope of the present disclosure.

1. A cosmetic composition comprising, in a cosmetically acceptable medium:
(a) at least one polyene;
(b) at least one polyether-modified silicone;
(c) at least one siloxysilicate resin; and
(d) at least one non-emulsifying silicone elastomer.

2. The cosmetic composition according to claim 1, wherein the at least one polyene (a) is selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof.

3. The cosmetic composition according to claim 1, wherein the at least one polyene (a) is selected from the group consisting of polybutene, polyisobutene, polyisoprene, polypentene, polypropylene, and mixtures thereof.

4. The cosmetic composition according to claim 1, wherein the at least one polyether-modified silicone (b) is selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof.

5. The cosmetic composition according to claim 4, wherein the at least one polyether-modified silicone (b) is selected from the group consisting of PEG-3 Dimethicone, PEG-8 Dimethicone, PEG-10 Dimethicone, PEG-12 Dimethicone, and mixtures thereof.

6. The cosmetic composition according to claim 1, wherein the at least one siloxysilicate resin (c) is selected from the group consisting of Trimethylsiloxysilicate, Diisostearoyl Trimethylolpropane Siloxysilicate, and mixtures thereof.

7. The cosmetic composition according to claim 1, wherein the at least one non-emulsifying silicone elastomer (d) is selected from Vinyl Dimethicone/Methicone Silsesquioxane Crosspolymer.

8. The cosmetic composition according to claim 1, wherein the at least one polyene (a) is present at a concentration ranging from about 0.01 to about 5 wt. % based on the total weight of the composition.

9. The cosmetic composition according to claim 1, wherein the at least one polyether-modified silicone (b) is present at a concentration ranging from about 0.01 to about 5 wt. % based on the total weight of the composition.

10. The cosmetic composition according to claim 1, wherein the at least one siloxysilicate resin (c) is present at a concentration ranging from about 0.1 to about 25 wt. % based on the total weight of the composition.

11. The cosmetic composition according to claim 1, wherein the at least one non-emulsifying silicone elastomer (d) is present at a concentration ranging from about 0.1 to about 10 wt. % based on the total weight of the composition.

12. The cosmetic composition according to claim 1 comprising, in a cosmetically acceptable medium:
(a) at least one polyene selected the group consisting of from butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) at least one siloxysilicate resin selected from the group consisting of Trimethylsiloxysilicate, Diisostearoyl Trimethylolpropane Siloxysilicate, and mixtures thereof; and
(d) at least one non-emulsifying silicone elastomer selected from Vinyl Dimethicone/Methicone Silsesquioxane Crosspolymer.

13. The cosmetic composition according to claim 1 comprising, in a cosmetically acceptable medium:
(a) at least one polyene selected from the group consisting of polybutene, polyisobutene, polyisoprene, polypentene, polypropylene, and mixtures thereof;
(b) at least one polyether-modified silicone selected from the group consisting of PEG-3 Dimethicone, PEG-8 Dimethicone, PEG-10 Dimethicone, PEG-12 Dimethicone, and mixtures thereof;
(c) at least one siloxysilicate resin selected from the group consisting of Trimethylsiloxysilicate, Diisostearoyl Trimethylolpropane Siloxysilicate, and mixtures thereof; and
(d) at least one non-emulsifying silicone elastomer selected from Vinyl Dimethicone/Methicone Silsesquioxane Crosspolymer.

14. The cosmetic composition according to claim 1 comprising, in a cosmetically acceptable medium, based on the total weight of the composition:
(a) about 0.01 to 5 wt. % of at least one polyene;
(b) about 0.01 to 5 wt. % of at least one polyether-modified silicone;
(c) about 0.1 to 25 wt. % of at least one siloxysilicate resin; and
(d) about 0.1 to 10 wt. % of at least one non-emulsifying silicone elastomer.

15. The cosmetic composition according to claim 1 comprising, in a cosmetically acceptable medium, based on the total weight of the composition:
(a) about 0.01 to 5 wt. % of at least one polyene selected from the group consisting of butene/propylene copolymer, butylene/ethylene copolymer, butylene/ethylene/propylene copolymer, decene/butene copolymer, ethylene/octene copolymer, ethylene/propylene copolymer, hydrogenated poly(C6-12 olefin), hydrogenated poly(C6-14 olefin), hydrogenated poly(C6-20 olefin), hydrogenated polybutene, hydrogenated polydecene, hydrogenated polydodecene, hydrogenated polyisobutene, isobutylene/isoprene copolymer, isoprene/pentadiene copolymer, polybutene, poly(C4-12 olefin), poly(C6-14 olefin), poly(C20-28 olefin), poly(C30-45 olefin), polydecene, polyethylene, polyisobutene, polyisoprene, polypentene, polypropylene, and mixtures thereof;
(b) about 0.01 to 5 wt. % of at least one polyether-modified silicone selected from the group consisting of PEG-11 Methyl Ether Dimethicone, PEG-32 Methyl Ether Dimethicone, PEG/PPG-20/22 Butyl Ether Dimethicone, PEG-3 Dimethicone, PEG-8 Dimethicone, PEG-10 Dimethicone, PEG-12 Dimethicone, Cetyl PEG/PPG-10/1 Dimethicone, PEG-9 Polydimethylsiloxyethyl Dimethicone, and mixtures thereof;
(c) about 0.1 to 25 wt. % of at least one siloxysilicate resin selected from the group consisting of Trimethylsiloxysilicate, Diisostearoyl Trimethylolpropane Siloxysilicate, and mixtures thereof; and
(d) about 0.1 to 10 wt. % of at least one non-emulsifying silicone elastomer selected from Vinyl Dimethicone/Methicone Silsesquioxane Crosspolymer.

16. The cosmetic composition according to claim 1 comprising, in a cosmetically acceptable medium, based on the total weight of the composition:
(a) about 0.05 to 3 wt. % of at least one polyene;
(b) about 0.05 to 3 wt. % of at least one polyether-modified silicone;
(c) about 1 to 20 wt. % of at least one siloxysilicate resin; and
(d) about 0.5 to 5 wt. % of at least one non-emulsifying silicone elastomer.

17. The cosmetic composition according to claim 1 comprising, in a cosmetically acceptable medium:
(a) about 0.05 to 3 wt. % of at least one polyene selected from the group consisting of polybutene, polyisobutene, polyisoprene, polypentene, polypropylene, and mixtures thereof;
(b) about 0.05 to 3 wt. % of at least one polyether-modified silicone selected from the group consisting of PEG-3 Dimethicone, PEG-8 Dimethicone, PEG-10 Dimethicone, PEG-12 Dimethicone, and mixtures thereof;
(c) about 1 to 20 wt. % of at least one siloxysilicate resin selected from the group consisting of Trimethylsiloxysilicate, Diisostearoyl Trimethylolpropane Siloxysilicate, and mixtures thereof; and
(d) about 0.5 to 5 wt. % of at least one non-emulsifying silicone elastomer selected from Vinyl Dimethicone/Methicone Silsesquioxane Crosspolymer.

18. A method for providing a long-wearing durable fill on the lips comprising applying to the lips a composition as defined in claim 1.

19. A method for applying color cosmetics to lips comprising applying to the lips a composition as defined in claim 1.
